(19) United States
(12) Patent Application Publication
(10) Pub. No.: US 2004/0192946 A1
(43) Pub. Date: Sep. 30, 2004

(54) Method for the production of propylene oxide

(76) Inventors: Joaquim H Teles, Otterstadt (DE); Alwin Rehfinger, Mutterstadt (DE); Anne Berg, Merksem (BE); Peter Rudolf, Ladenburg (DE); Robert Rieber, Mannheim (DE); Peter Bassler, Viernheim (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/485,104

(22) PCT Filed: Jul. 30, 2002
(86) PCT No.: PCT/EP02/08487

(30) Foreign Application Priority Data
Aug. 1, 2001 (DE) 101 37 543.3

(51) Int. Cl. C07D 301/12

(52) U.S. Cl. 549/531

(57) ABSTRACT

A process for preparing propylene oxide comprises at least the following steps:

(a) propene is reacted with hydrogen peroxide in a solvent in the presence of a suitable catalyst to give a mixture (M0) comprising propylene oxide, solvent, unreacted propene, unreacted hydrogen peroxide and oxygen,

(b) the propylene oxide is separated off from the mixture (M0) so as to give a mixture (M1) comprising at least unreacted propene and oxygen, and

(c) the mixture (M1) is utilized.

The present invention relates to a process in which propylene oxide is prepared from hydrogen peroxide and propane and in which a mixture comprising unreacted propene and oxygen is obtained and subsequently utilized.

In many processes for preparing propylene oxide from propene as starting material, efforts are made to recover the propene which has not been reacted in the process as completely as possible from the product mixture, if necessary purify it and reuse it as starting material in the process. In one possible embodiment, this product mixture is subjected to a distillation in which the unreacted propene is removed together with compounds having a boiling point lower than that of propene from the product mixture. The unreacted propene is subsequently separated off from this low-boiling fraction, worked up if appropriate and returned to the process. Such processes are described in, for example, DE 10001401.1.

However, a problem which frequently arises in the recovery of propene is that propene and oxygen can be present in concentrations which lead to formation of ignitable mixtures. Separating off the propene therefore presents a serious safety risk.

To work up or separate off the propene safely, it is therefore necessary to avoid the formation of an ignitable mixture of propene and oxygen. For this purpose, it is proposed in, for example, EP-B 0 719 768 that the separation by distillation of the unreacted propene from the low-boiling fraction be carried out in a so-called absorption zone by means of a suitable absorption medium with additional introduction of an inert gas into this absorption zone so as to dilute the oxygen to a concentration at which the mixture is no longer ignitable.

In a further patent application DE 10001401.1, which likewise relates to a process for preparing propylene oxide, formation of an ignitable mixture in the separation of unreacted propene from a mixture comprising propene and oxygen is avoided by the following method: propene and a hydroperoxide are firstly reacted in a solvent in the presence of a titanium silicate catalyst to form propylene oxide and give a mixture which comprises unreacted propene and oxygen together with further components. Oxygen is removed from this mixture by a catalytic process to give a further mixture comprising propene, and propene is subsequently separated by distillation from this further mixture and returned to the process as starting material.

A further possibility for a safe work-up is described in a further patent application by the present applicant. This application relates to a process for preparing propylene oxide in which a mixture comprising unreacted propene and oxygen is separated off from the product mixture in such a way that it is not ignitable. The nonignitability of this mixture is achieved by the concentration of oxygen in the mixture being less than 12% by volume.

The safe work-up of the low-boiling fraction comprising unreacted propene accordingly requires an increased outlay in terms of apparatus and, associated directly therewith, an increased energy consumption. As a result, the overall process, i.e. the preparation of propylene oxide together with the recovery and recirculation of unreacted propene, is frequently energy-inefficient. The recovery of unreacted propene for the purpose of returning it to the process therefore frequently does not appear to be economically viable when viewed in terms of the overall economics of the process.

However, there is of course a need, especially at the present time, not only from the point of view of conserving resources, to work up each partly unreacted starting material or intermediate which is not directly utilizable in the further process in an economically worthwhile fashion and thus to make the overall process more economical and thus also more competitive.

It is an object of the present invention to provide a process for preparing propylene oxide which is more efficient than the processes of the prior art.

We have found that this object is achieved by a process for preparing propylene oxide, which comprises at least the following steps:

(a) propene is reacted with hydrogen peroxide in a solvent in the presence of a suitable catalyst to give a mixture (M0) comprising at least propylene oxide, solvent, unreacted propene, unreacted hydrogen peroxide and oxygen,

(b) the propylene oxide is separated off from the mixture (M0) so as to give a mixture (Ml) comprising at least unreacted propene and oxygen, and

(c) the mixture (M1) is utilized.

Step (a) of the process of the present invention can be carried out by all methods known to those skilled in the art for this reaction, in particular in accordance with the patent applications DE 19835907.1, DE 19936547.4, DE 10015246.5 and DE 10032885.7.

The reaction of propene with hydrogen peroxide in a solvent in the presence of a suitable catalyst to give a mixture (M0) is preferably carried out in at least one shell-and-tube reactor.

In the process of the present invention, it is in principle possible to use all solvents which appear suitable to a person skilled in the art. Examples of solvents which can be used are:

water,

alcohols, preferably lower alcohols, more preferably alcohols having less than 6 carbon atoms, for example methanol, ethanol, propanols, butanols, pentanols,

diols or polyols, preferably those having less than 6 carbon atoms,

ethers such as diethyl ether, tetrahydrofuran, dioxane, 1,2-diethoxyethane, 2-methoxyethanol,

esters such as methyl acetate or butyrolactone,

amides such as dimethylformamide, dimethylacetamide, N-methylpyrrolidone,

ketones such as acetone,

nitriles such as acetonitrile

or mixtures of two or more of the abovementioned compounds.

Methanol is preferably used as solvent in the process of the present invention.

Catalysts which can be used in step (a) of the process of the present invention are in principle all catalysts known to those skilled in the art for such a reaction, preferably zeolite catalysts.

Preference is given to zeolites in which iron, titanium, vanadium, chromium, niobium or zirconium is present.

Specific examples to be named are titanium-, germanium-, tellurium-, vanadium-, chromium-, niobium-, zirconium-containing zeolites having a pentasil zeolite structure, in particular the types which can be assigned on the basis of the X-ray diffraction patterns to the ABW, ACO, AEI, AEL, AEN, AET, AFG, AFI, AFN, AFO, AFR, AFS, AFT, AFX, AFY, AHT, ANA, APC, APD, AST, ATN, ATO, ATS, ATT, ATV, AWO, AWW, BEA, BIK, BOG, BPH, BRE, CAN, CAS, CFI, CGF, CGS, CHA, CHI, CLO, CON, CZP, DAC, DDR, DFO, DFT, DOH, DON, EAB, EDI, EMT, EPI, ERI, ESV, EUO, FAU, FER, GIS, GME, GOO, HEU, IFR, ISV, ITE, JBW, KFI, LAU, LEV, LIO, LOS, LOV, LTA, LTL, LTN, MAZ, MEI, MEL, MEP, MER, MFI, MFS, MON, MOR, MSO, MTF, MTN, MTT, MTW, MWW, NAT, NES, NON, OFF, OSI, PAR, PAU, PHI, RHO, RON, RSN, RTE, RTH, RUT, SAO, SAT, SBE, SBS, SBT, SFF, SGT, SOD, STF, STI, STT, TER, THO, TON, TSC, VET, VFI, VNI, VSV, WIE, WEN, YUG, ZON structures and to mixed structures derived from two or more of the abovementioned structures. It is also conceivable to use titanium-containing zeolites having the ITQ-4, SSZ-24, TTM-1, UTD-1, CIT-1or CIT-5 structure in the process of the present invention. Further titanium-containing zeolites which may be mentioned are those having the ZSM-48 or ZSM-12 structure.

For the purposes of the present invention, preference is given to using Ti zeolites having an MFI, MEL or mixed MFI/MEL structure. Further examples of preferred zeolites are the Ti-containing zeolite catalysts which are generally referred to as “TS-1”, “TS-2”, “TS-3”, and also Ti zeolites having a framework structure isomorphous with β-zeolite.

Accordingly, the process of the present invention, as described above, is particularly preferably carried out using a titanium silicalite catalyst, in particular a titanium silicalite catalyst having a TS-1 structure, as zeolite catalyst.

Further details regarding the catalysts which can be used, in particular zeolites, may be found in DE 10010139.2.

The mixture (M0) resulting from the reaction in step (a) comprises essentially the following components: propylene oxide as desired process product, solvent, water, unreacted hydroperoxide, unreacted propene and oxygen.

For the purposes of the present invention, it is of course also possible to use propene which contains up to 10% by weight of hydrocarbons other than propene.

For example, the propene used can contain up to 10% by weight of propane, ethane, ethylene, butane or butenes, either individually or as a mixture of two or more thereof.

In a further step (b), propylene oxide is separated from off from the mixture (M0) resulting from step (a) of the process of the present invention so as to give a mixture (M1) which comprises at least unreacted propene and oxygen.

The separation of propylene oxide from the mixture (M0) in step (b) of the process of the present invention can be carried out by any method known to those skilled in the art for such a separation.

Thus, further intermediate steps which are known to those skilled in the art and appear suitable in the context of a process for preparing propylene oxide can of course be interposed between steps (a) and (b).

The mixture arising from step (b) is then utilized in a further step of the process of the present invention, viz. step (c).

The mixture (M1) can be utilized in any manner known to a person skilled in the art. Thus, for example, it is possible to use the mixture (M1) as starting material in one of the following processes: acrylic acid production, acrylonitrile production, acrolein production and acetone production.

In addition, the mixture (M1) can also be used for energy recovery in step (c).

The present invention therefore also provides a process as described above in which the mixture (M1) is used for energy recovery in step (c).

In the process of the present invention, the gaseous mixture (M1) which has been separated off from the mixture (M0) in the above-described manner is for this purpose firstly passed to at least one further work-up apparatus. In this, the mixture (M1) is preferably admixed with further oxygen and subsequently burnt. The heat energy which is liberated in this way can, for example, be converted into an economically utilizable form of energy.

Accordingly, the present invention also provides a process as described above in which the energy liberated in step (c) is used for the generation of water vapor.

Thus, the heat of combustion obtained in step (c) of the process of the present invention is used for heating a fluid medium for the purpose of generating vapor. The vapor generated in this way can be used beneficially in a variety of ways in the abovementioned process.

One way of utilizing this vapor in an economically useful fashion is, for example, direct heating of apparatuses used in the process. Furthermore, the vapor can be converted into further forms of energy which can be utilized economically within the process, for example into mechanical or electrical energy by means of converters known for this purpose to those skilled in the art.

The mechanical or electrical energy obtained in this way is advantageously used in the process of the present invention for operating the apparatuses used in the process, so that an energy-efficient and thus environmentally friendly process is achieved.

The present invention therefore also provides a process as described above in which the water vapor generated is used as energy transfer medium for operating distillation columns in the process of the present invention.

1-6 (Canceled).

7. A process for preparing propylene oxide which comprises at least the following steps:
(a) propene is reacted with hydrogen peroxide in a solvent in the presence of a zeolite catalyst to give a mixture (M0) comprising at least propylene oxide, solvent, unreacted propene, unreacted hydrogen peroxide and oxygen,
(b) the propylene oxide is separated off from the mixture (M0) so as to give a mixture (M1) comprising at least unreacted propene and oxygen, and
(c) the mixture (M1) is utilized,
wherein during utilization according to (c) the propene comprised in mixture (M1) is not recirculated into the reaction according to (a).

8. A process as claimed in claim 7, wherein during utilization according to (c) the mixture (M1) is used for recovering energy and the energy liberated in step (c) is used for generating vapor.

9. A process as claimed in claim 8 wherein, in order to recover energy, the mixture (M1) is
(aa) admixed with further oxygen in at least one work-up apparatus,
(bb) the mixture resulting from (aa) is burnt, and
(cc) the heat energy resulting from (bb) is used for generating vapor.

10. A process as claimed in claim 9 wherein, according to (cc), water vapor is generated.

11. A process as claimed in claim 10 wherein the water vapor generated according to (cc) is used as energy transfer medium for operating distillation columns in a process for preparing propylene oxide which comprises at least the following steps:
(a) propene is reacted with hydrogen peroxide in a solvent in the presence of a zeolite catalyst to give a mixture (M0) comprising at least propylene oxide, solvent, unreacted propene, unreacted hydrogen peroxide and oxygen,
(b) the propylene oxide is separated off from the mixture (M0) so as to give a mixture (M1) comprising at least unreacted propene and oxygen, and
(c) the mixture (M1) is utilized,
wherein during utilization according to (c) the propene comprised in mixture (M1) is not recirculated into the reaction according to (a).

12. A process as claimed in claim 7 wherein during utilization according to (c) the mixture (M1) is used as starting material in one of the processes acrylic acid production, acrylonitril production, acrolein production and acetone production.
